(19) United States
(12) Patent Application Publication
(10) Pub. No.: US 2007/0162023 A1
(43) Pub. Date: Jul. 12, 2007

(54) Washer for pedicle screw

(76) Inventor: Charles C. Schock, North Little Rock, AR (US)

Correspondence Address: WRIGHT, LINDSEY & JENNINGS LLP, 200 WEST CAPITOL AVENUE, SUITE 2300, LITTLE ROCK, AR, 72201-3699, US

(21) Appl. No.: 11/486,983

(22) Filed: Jul. 14, 2006

Related U.S. Application Data
(60) Provisional application No. 60/699,512, filed on Jul. 15, 2005.
(60) Provisional application No. 60/748,329, filed on Dec. 7, 2005.

(51) Int. Cl. A61B 17/58

(52) U.S. Cl. 606/72

(57) ABSTRACT

A washer for use in combination with a pedicle screw of the type having a toggling connecting member. The washer has a face comprising a spherical annulus facing the bottom surface of the connecting member. The spherical annulus is geometrically a portion of a sphere having a center coincident with the center of rotation of the hemispherical head of the pedicle screw. The radius of the sphere containing the spherical annulus of the washer is the same radius as the sphere containing the circle where the washer and the connecting member touch when the pedicle screw is tightly screwed to the bone. The connecting member is thereby able to toggle even with the screw firmly seated to the bone. In an alternative embodiment, the washer is provided with a low narrow profile to a avoid an significant increase in the height or width of the pedicle screw alone. A router is described for flattening a portion of bone surface to receive the washer.

### CROSS-REFERENCE TO RELATED APPLICATIONS

This application claims the benefit of U.S. Provisional Application No. 60/699,512 filed Jul. 15, 2005, the disclosure of which is incorporated herein by reference.

### STATEMENT REGARDING FEDERALLY SPONSORED RESEARCH OR DEVELOPMENT

Not applicable.

### BACKGROUND OF THE INVENTION

The present invention relates to washer for use with a polyaxial pedicle screw.

There are conditions of the spine involving displacements or deformities of the vertebrae that may be corrected by surgical means employing plates, rods and the like that are fastened to the vertebrae and that serve to fix the vertebrae in desired positions. Such devices are typically affixed to the vertebrae by screws into the pedicle of the vertebra.

Polyaxial pedicle screws generally comprise a screw with a hemispherical head and a connecting member which includes means for affixing a rod. The hemispherical head of the screw is mounted in the connecting member such that the screw can be adjusted angularly with respect to the connecting member. The connecting member is thereby allowed to toggle to some degree to accommodate the connecting member to the position of the rod. Therefore one vertebra can be linked more easily to another vertebra with a rod affixed between two connecting members, each is which is affixed to a respective vertebra by one or more pedicle screws. Polyaxial pedicle screws that do not have hemispherical heads are also known. The term “polyaxial pedicle screw” as used herein is intended to encompass all pedicle screws having connecting members that have some ability to toggle about a center of rotation.

A common type of connecting member, such as that made by Synthes (U.S.A.) has a tulip shaped connecting member; i.e., a generally cylindrically-shaped body having a flat portion on the bottom adjacent to the screw and a rounded transition between the flat bottom portion and the generally straight cylindrical side wall. This type of connecting member suffers from a significant limitation. The pedicle screw cannot be screwed tightly to the bone without eliminating its ability to toggle. Once the flat portion is in contact with and tightly screwed to the bone, the connecting member is unable to rotate about the hemispherical head of the pedicle screw.

In order for the connecting member to be able to toggle, the screw must be loosely affixed to the bone, which increases its potential for eventual loosening from the bone and negating the purpose of affixing the vertebrae into a desired position.

These and other problems of the prior art are addressed by the present invention as described following.

### BRIEF SUMMARY OF THE INVENTION

The present invention is a washer for use in combination with a pedicle screw of the type described above. The washer has a body provided with a face comprising a spherical annulus facing the bottom surface of the connecting member. The washer's opposite face is flat for contact with the bone. In use, the spherical annulus touches the bottom surface of the connecting member along a circle. The spherical annulus is geometrically a portion of a sphere having a center coincident with the center of rotation of the head of the pedicle screw. The sphere contains the circle where the washer and the connecting member touch; i.e., the radius of the sphere containing the spherical annulus of the washer is the same radius as the sphere containing the circle where the washer and the connecting member touch when the pedicle screw is tightly screwed to the bone.

By this arrangement, the connecting member is able to toggle even with the screw firmly seated to the bone since the connecting member is able to rotate between the head of the screw and the spherical annulus of the washer.

### BRIEF DESCRIPTION OF THE DRAWINGS

FIG. 1 is an elevation view of a polyaxial pedicle screw showing how a rod is affixed in the slot of the connecting member by a set screw. The rod is shown in cross section. The center of rotation of the connecting member in this and the following figures is indicated by C.

FIG. 2 is an elevation view of the pedicle screw of FIG. 1 showing a rod whose desired position is offset from the slot of the connecting member.

FIG. 3 is an elevation view of the pedicle screw of FIG. 2 showing how the connecting member may be toggled about the center C to align with the desired position of the rod.

FIG. 4 is an elevation view of a pedicle screw affixed tightly to the bone so that toggling of the connecting member is not possible.

FIG. 5 is an elevation view of the pedicle screw of FIG. 4 showing how potential toggling is blocked by the bone when the pedicle screw is tightly screwed to the bone.

FIG. 6 is an elevation view of a pedicle screw loosely screwed to the bone.

FIG. 7 is an elevation view of the pedicle screw of FIG. 6 showing that toggling of the connecting member is possible if the pedicle screw is loosely screwed to the bone.

FIG. 8 is an elevation view of a pedicle screw used in combination with the washer of the present invention. The washer is shown in cross-section. The pedicle screw is tightly screwed to the bone thus providing firm seating against the bone.

FIG. 9 is a perspective view of the washer of the present invention showing the face comprising a spherical annulus.

FIG. 10 is an elevation view of a pedicle screw in combination with the washer of the present invention showing the hemispherical head of the screw in a cross-section view of the connecting member. The washer is shown in cross-section.

FIG. 11 is an elevation view of the pedicle screw of FIG. 10 showing the connecting member in a toggled position.

FIG. 12 is an elevation view of a polyaxial pedicle screw showing an alternative embodiment of the washer of the present invention. In this alternative embodiment the washer is provided with a low narrow profile so as to avoid any significant increase in the height or width over that of the connecting member of the pedicle screw alone.

FIG. 13 is an elevation view of a router for use with the washer of the present invention where the open-sided cylinder of the router is shown in partial cross section enclosing a tap.

FIG. 14 is a bottom plan view of the router of FIG. 13 showing a serrated bottom surface of the open-sided cylinder for flattening a portion of bone surface to support the washer.

### DETAILED DESCRIPTION OF THE INVENTION

The preferred embodiments of the invention are described herein with reference to FIGS. 1-14.

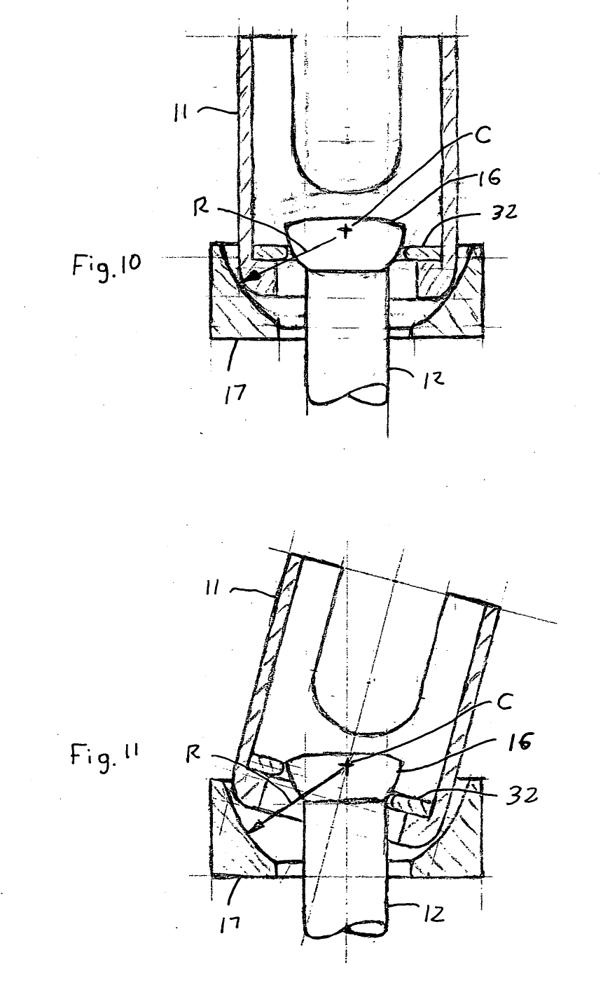

FIG. 1 shows a prior art polyaxial pedicle screw 10 comprising a connecting member 11 and screw 12. The connecting member 11 has an end opposite to the screw 12, a transverse slot 13 in which a rod 14 may be affixed by a set screw 15. The screw 12 has a hemispherical head 16 as shown in FIGS. 10 and 11 which is received in a seat 32 such that the screw 12 is able to rotate to a limited degree about a center of rotation C. The seat 32 is disk-shaped with a central opening. The hemispherical head 16 bears against the circular central opening when the pedicle screw 10 is tightly affixed to the bone 18.

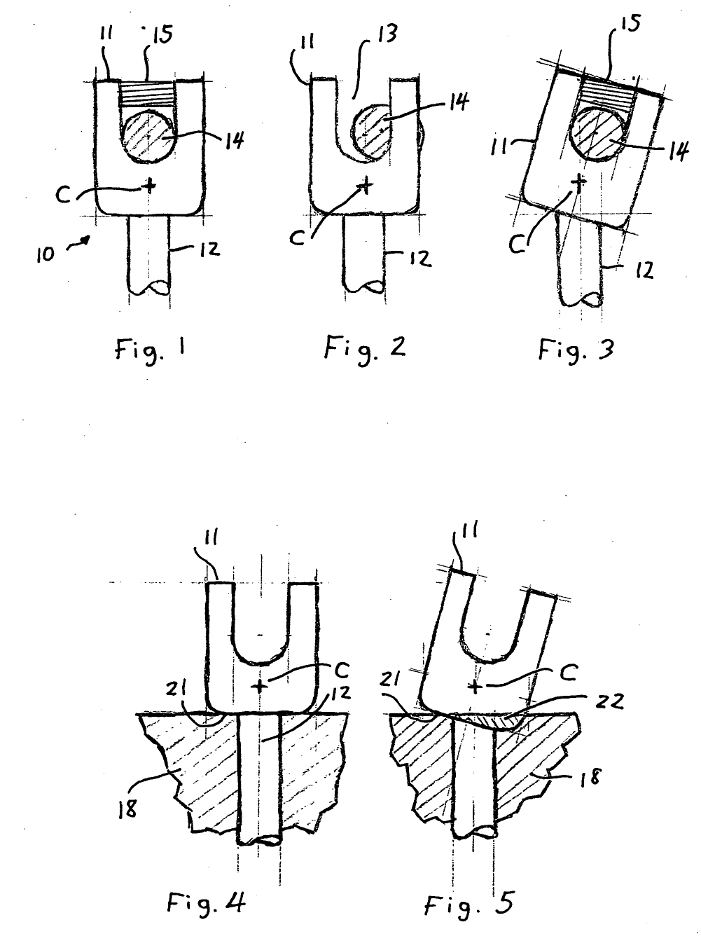

FIG. 2 illustrates the situation where the polyaxial pedicle screw 10 is not aligned with the rod 14 such that the rod 14 is unable to seat in the slot 13 of the connecting member 11.

By toggling the connecting member 11, the offset rod 14 can be aligned with and received into the slot 13, where it may be affixed with the set screw 15 as shown in FIG. 3.

Such toggling action is only possible when the polyaxial pedicle screw 10 is not affixed tightly to the bone 18 as shown in FIG. 4. The flat bottom portion 21 of the connecting member 11, when seated tightly to the bone 18, prevents the connecting member 11 from rotating as shown in FIG. 5 since a toggling of the connecting member is now blocked by the area of bone 22 shown in FIG. 5.

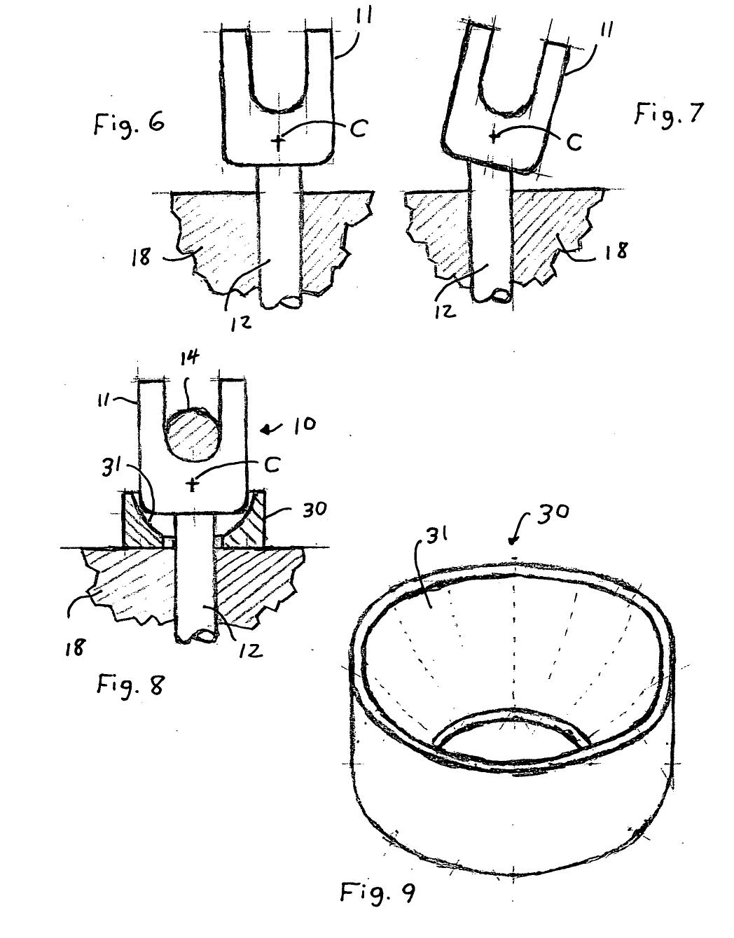

When the pedicle screw 10 is loosely screwed to the bone 18 of, e.g., a vertebra, as shown in FIG. 6, the connecting member 11 can be moved to a toggled position as shown in FIG. 7.

The present invention solves the problem of toggling the connecting member 11 when the pedicle screw 10 is tightly affixed to the bone 18. FIG. 8 shows the pedicle screw 10 used in combination with the washer 30 of the present invention. The washer 30 is shown in cross-section and the pedicle screw 10 is shown tightly screwed to the bone 18. As shown in FIGS. 9-11, the washer 30 has a body with a face comprising a spherical annulus 31 and an opposite flat face 17. The washer is provided with a central hole for receiving the screw 10. The hole is desirably large enough to accommodate some movement of the washer with respect to the screw 10. As used in combination with the pedicle screw 10, the spherical annulus 31 of the washer 30 faces the flat bottom surface 21 of the connecting member 11. The spherical annulus 31 touches the bottom surface 21 along a circle. The spherical annulus 31 is geometrically a portion of a sphere having a center coincident with the center of rotation C of the hemispherical head 16 of the pedicle screw 10 when the pedicle screw 10 is tightly screwed to the bone 18. This geometrical sphere contains the circle or portion of a circle where the washer 30 and the connecting member 11 touch; i.e., the radius R of the sphere containing the spherical annulus 31 of the washer 30 is the same radius as the sphere containing the circle or portion of a circle where the washer 30 and the connecting member 11 touch when the pedicle screw 10 is tightly screwed to the bone 18.

By this arrangement, the connecting member 11 is able to toggle even with the pedicle screw 10 firmly seated to the bone 18 as shown in FIG. 11 since the connecting member 11 is free to rotate between the hemispherical head 16 of the screw 12 and the spherical annulus 31 of the washer 30. FIG. 11 shows the connecting member 11 in a toggled position with the pedicle screw 10 tightly screwed to the bone 18.

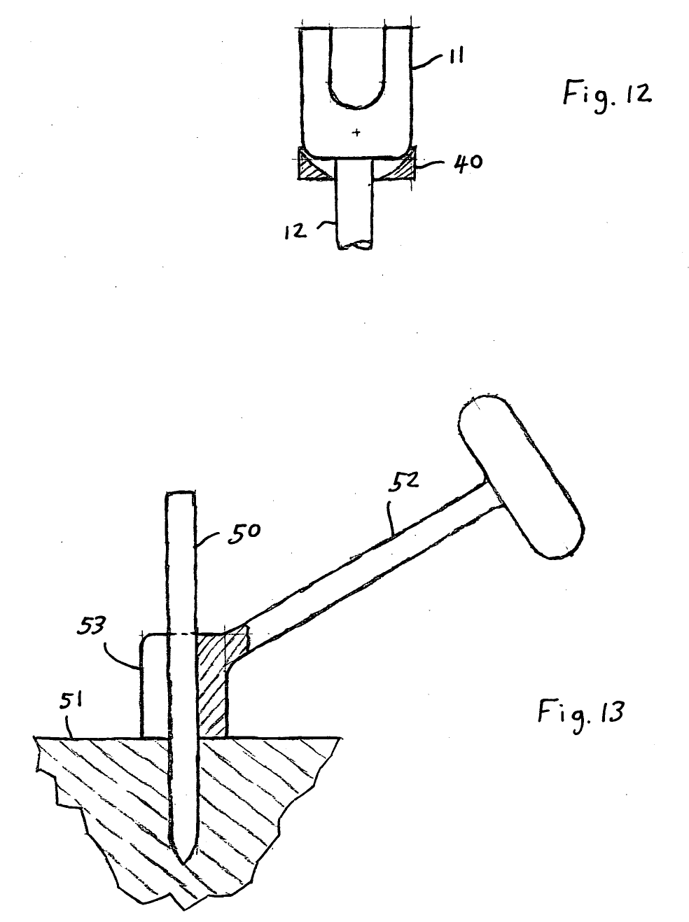

FIG. 12 is an elevation view of a polyaxial pedicle screw showing an alternative embodiment of the washer of the present invention. In this alternative embodiment the washer 40 is provided with a low narrow profile so as to avoid any significant increase in the height or width over that of the connecting member 11 of the pedicle screw alone. It is desirable that the washer 40 does not extend substantially beyond the width of the connecting member 11 and in any event no more than is necessary to maintain the structural integrity and dependability of the washer 40 in various toggling positions.

It is desirable that the washer 30, 40 be made from materials that are biocompatible. Further, it is desirable that the materials be the same as, or compatible with, the materials from which the pedicle screw and connecting member are made. In some applications, it may also be desirable that the washer 30, 40 be coated with a different material for lubrication, corrosion resistance or other purposes. For example, the flat face 17 may be provided with a coating to facilitate osseointegration with the bone surface. Such a coating may be formed on a titanium washer body by first applying a titanium plasma spray and then coating with hydroxylapatite.

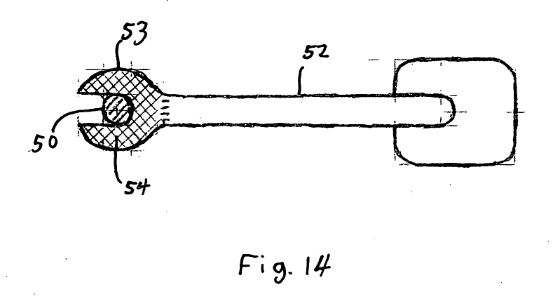

FIG. 13 is an elevation view of a router for use with the washer of the present invention. It is desirable that the washer be well seated against the bone. To this end it is desirable that the portion of the bone surface 51 which receives the washer be substantially flat. In order to attach the pedicle screw, a tap 50 is first used to prepare a hole in the bone 51 to receive the screw 12 of the pedicle screw 10. While the tap 50 is engaged with the bone 51, the router is used to flatten the surface of the bone 51 around the point where the tap 50 enters the bone 51. As shown in FIGS. 13 and 14, the router comprises an open-sided cylinder 53 which fits around the tap 50. In FIG. 13, the open-sided cylinder 53 of the router is shown in partial cross section enclosing the tap 50. By means of the handle 52, the router is moved back and forth around the tap 50 in a reciprocating circular motion.

As seen in FIG. 14 the open-sided cylinder 53 is provided with a serrated bottom surface 54. The serrated bottom surface is held in contact with the surface of the bone 51 while the router is moved back and forth around the tap 50. The reciprocating circular motion of the serrated bottom surface 54 acts to flatten the portion of bone surface with which it is in contact. The flattened bone surface provides a substantially flat surface against which to seat the washer. Optionally, the router may also be provided with a serrated side surface in order to assist in carving out a cylindrical depression to receive the washer and the connecting member.

The present invention has been described with reference to certain preferred and alternative embodiments that are intended to be exemplary only and not limiting to the full scope of the present invention as set forth in the appended claims. Although the preferred embodiments of the present invention have been described with respect to a pedicle screw, the present invention is not so limited and may be employed in any orthopedic procedures where toggling of a connecting member is desirable.

What is claimed is:

1. A washer for use with a screw for attachment of an orthopedic device to a bone, the screw being of the type having a connecting member that toggles about a center of rotation, comprising:
a body with a face comprising a spherical annulus wherein a sphere containing said spherical annulus has a radius substantially equivalent to a radius of a sphere containing at least a portion of a circle where said washer and the connecting member contact when the screw is tightly screwed to the bone.

2. The washer of claim 1, further comprising a substantially flat opposite face.

3. The washer of claim 1, further comprising a central hole for receiving the screw.

4. The washer of claim 1, wherein a width of the washer is not substantially greater than a width of the connecting member.
